SEQUENCE LISTING (1 sequence)

SEQ ID NO: 1 - protein, 28 aa, Artificial Sequence
  Thymosin alpha 1 immunomodulatory thymic peptide
SDAAVDTSSEITTKDLKEKKEVVEEAEN

(19) United States
(12) Patent Application Publication
(10) Pub. No.: US 2010/0221274 A1
(43) Pub. Date: Sep. 2, 2010

(54) METHOD OF ADMINISTERING A THYMOSIN ALPHA 1 PEPTIDE

(75) Inventors: ALFRED RUDOLPH, LOS ALTOS HILLS, CA (US); CYNTHIA W. TUTHILL, MENLO PARK, CA (US)

Correspondence Address: COOLEY LLP;ATTN: Patent Group, Suite 1100, 777 - 6th Street, NW, WASHINGTON, DC, 20001, US

(73) Assignee: SCICLONE PHARMACEUTICALS, INC., FOSTER CITY, CA (US)

(21) Appl. No.: 12/775,986

(22) Filed: May 7, 2010

Related U.S. Application Data
(63) Continuation of application No. 10/493,848, filed on Nov. 10, 2004, now abandoned.
(60) Provisional application No. 60/330,874, filed on Nov. 1, 2001.

(51) Int. Cl. A61K 39/00; A61P 37/04

(52) U.S. Cl. 424/185.1

(57) ABSTRACT

A Thymosin alpha 1 (TA1) peptide is administered to a patient in need of immune stimulation so as to substantially continuously maintain an immune stimulating-effective amount of the TA1 peptide in the patient. The administration method may be by continuous infusion.

### CROSS-REFERENCE TO RELATED APPLICATIONS

This application is a continuation of U.S. application Ser. No. 10/493,848 with a 371 filing date of Nov. 10, 2004 which is a national phase application of International Application No. PCT/US02/35093 filed Nov. 1, 2002 which claims priority to U.S. Provisional Patent Application Ser. No. 60/330,874, filed on Nov. 1, 2001, the contents of which are hereby incorporated by reference in their entirety for all purposes.

### FIELD OF THE INVENTION

The present invention relates to a method of administering a Thymosin alpha 1 peptide.

### DESCRIPTION OF THE BACKGROUND ART

Thymosin alpha 1 (sometimes referred to as TA1) is a 28-amino acid thymic peptide with immunomodulatory properties, homologous to a natural product originally isolated from thymosin fraction 5 of calf thymus. Its biological effects include augmentation of T lymphocyte function and include modulation of interleukin-2 (IL-2), stimulation of interferon-γ production, induction of T lymphocytes and NK cell activity, and stimulation of thymopoiesis. Thymosin alpha 1 also has been shown to up-regulate MHC Class I expression.

Thymosin alpha 1 has previously been suggested for use in certain treatments of cancer, Hepatitis B and C, HIV, etc., e.g., by subcutaneous injection twice weekly. There remains a need in the art for improved methods of administering Thymosin alpha 1.

### SUMMARY OF THE INVENTION

In accordance with the present invention, a method of administering a Thymosin alpha 1 (TA1) peptide to a patient in need of immune stimulation, comprises administering the TA1 peptide to the patient so as to substantially continuously maintain an immune stimulating-effective amount of the TA1 peptide in the patient during a treatment period of at least about six hours.

### DETAILED DESCRIPTION OF THE INVENTION

The present invention is based on a discovery that maintaining immune stimulating-effective amounts of a TA1 peptide in a patient's circulatory system during a treatment period provides a substantial improvement in the immune stimulating effect of the TA1 peptide.

The invention is applicable to TA1 peptides including naturally occurring TA1 as well as synthetic TA1 and recombinant TA1 having the amino acid sequence of naturally occurring TA1, amino acid sequences substantially similar thereto, or an abbreviated sequence form thereof, and their biologically active analogs having substituted, deleted, elongated, replaced, or otherwise modified sequences which possess bioactivity substantially similar to that of TA1, e.g., a TA1 derived peptide having sufficient amino acid homology with TA1 such that it functions in substantially the same way with substantially the same activity as TA1.

Because the plasma half-life of subcutaneously injected TA1 is only about two hours, according to one embodiment, a TA1 peptide such as TA1 is administered to a patient in need of immune stimulation so as to substantially continuously maintain an immune stimulating-effective amount of the TA1 peptide in the patient's circulatory system during a substantially longer treatment period. Although much longer treatment periods are contemplated in accordance with the present invention, embodiments of the invention include substantially continuously maintaining an immune stimulating-effective amount of the TA 1 peptide in the patient's circulatory system during treatment periods of at least about 6, 10, 12 hours, or longer. In other embodiments, treatment periods are for at least about a day, and even for a plurality of days, e.g., a week or longer. However, it is contemplated that treatments, as defined above, in which immune stimulating-effective amounts of the TA1 peptide are substantially continuously maintained in the patient's circulatory system, may be separated by non-treatment periods of similar or different durations.

In accordance with one embodiment, the TA1 peptide is continuously infused into a patient, e.g., by intravenous infusion, during the treatment period, so as to substantially continuously maintain an immune stimulating-effective amount of the TA1 peptide in the patient's circulatory system. The infusion may be carried out by any suitable means, such as by minipump.

Alternatively, an injection regimen of the TA1 peptide can be maintained so as to substantially continuously maintain an immune stimulating-effective amount of the TA1 peptide in the patient's circulatory system. Suitable injection regimens may include an injection every 1, 2, 4, 6, etc. hours, so as to substantially continuously maintain the immune stimulating-effective amount of the Thymosin alpha 1 peptide in the patient's circulatory system during the treatment period.

Although it is contemplated that during continuous infusion of the TA1 peptide, administration will be for a substantially longer duration, according to one embodiment the continuous infusion of the TA1 peptide is for a treatment period of at least about 1 hour. More preferably, continuous infusion is carried out for longer periods, such as for periods of at least about 6, 8, 10, 12 hours, or longer. In other embodiments, continuous infusion is for at least about one day, and even for a plurality of days such as for one week or more.

Immune stimulating-effective amounts of a TA1 peptide may be substantially continuously maintained in a patient's circulatory system by administering the TA1 peptide to the patient at a rate within a range of about 0.0001-0.1 mg/hr/Kg patient body weight. Preferred administration rates are within a range of about 0.0003-0.03 mg/hr/Kg patient body weight.

In preferred embodiments, the TA1 peptide is present in a pharmaceutically acceptable liquid carrier, such as water for injection, saline in physiological concentrations, or similar.

The invention may be utilized for treatment of any patient in need of immune stimulation, including cancer patients, HIV patients, and patients having various forms of hepatitis, including Hepatitis B and Hepatitis C. For example, the invention may be utilized to promote bone marrow recovery in cancer patients following chemotherapy. The invention may be particularly useful for addition of TA1 to chemoimmunotherapy for increased survival in melanoma and hepatocellular carcinoma (HCC) patients, and for reduction of haematological toxicity in lung cancer.

In the following examples, which are not intended to be limiting, a continuous infusion of TA1 (N-acetyl-Ser-Asp-Ala-Ala-Val-Asp-Thr-Ser-Ser-Glu-Ile-Thr-Thr-Lys-Asp-Leu-Lys-Glu-Lys-Lys-Glu-Val-Val-Glu-Glu-Ala-Glu-Asn-OH) (SEQ ID NO:1) was evaluated in a cancer therapy model with the use of surgically implanted osmotic minipumps, which deliver fluids at a constant flow rate for 5 days. Rats were given 5-fluorouracil (5-FU) to cause immune suppression, and then treated with injected or infused TA1 8 days later (the nadir of white cell count after 5-FU). Treatment groups, 8 rats each, were: control (minipumps with saline); low dose TA1 (0.2 mg/Kg sc injection; empty minipumps); high dose TA1 (3.5 mg/Kg sc injection; empty minipumps); and high dose infused TA1 (3.5 mg/Kg infused by minipumps). Immune parameters were determined at baseline and 8 days after 5-FU treatment (day 1 of TA1 treatment), and also at 5, 12, 20, and 27 days after TA1 treatment.

### EXAMPLE 1

10 week old rats, weighing 250-300 g, received 100 mg/kg 5-fluorouracil (5-FU) for immune suppression.

8 days after 5-FU treatment, rats were randomly assigned to one of the following groups (n=8):
Control (saline in minipump)
Low dose TA1, injected s.c. at 0.2 mg/Kg (with empty minipumps)
High dose TA1, injected s.c. at 3.5 mg/Kg (with empty minipumps)
Continuous infusion TA1, provided by minipump at 3.5 mg/Kg/5 days

Immune parameters were determined at baseline and 8 days after 5-FU treatment (day 1 of TA1 treatment), and also at 5, 12, 20, and 27 days after TA1 treatment.

The evaluations included NK activity (LDH released from YAC-1 cells after 4 h exposure to PBMC), total leukocyte number (judged by physical cytofluorimetric parameters, after verifying the specificity by monoclonal antibody), total lymphocyte number (CD3+ by flow cytometry), and activated lymphocytes (CD25+CD3+ by flow cytometry).

NK activity was 42±5% at baseline and was depressed to 9±2% after 5-FU. Low dose TA1 treatment lead to a significant recovery of NK activity after 12 days, while high dose TA1 achieved significant recovery in only 5 days. Continuous infusion of TA1, however, was able to double the response at 5 days, to 32±4% (versus 16±2 for high dose injected, 12±3 low dose injected, and 11±1 control). Only animals treated with TA1 by continuous infusion had a complete recovery of NK activity to baseline levels.

Total white blood cell count, as determined by morphology, was depressed from 14,590±2,071 cells/mm³ to 2,597±582 after treatment with 5-FU. Low or high dose TA1 treatment by injection trended towards a sooner increase in recovery compared to untreated animals. Continuous infusion of TA1, however, provided statistically significant and complete recovery to baseline levels after only 5 days.

Activated lymphocytes (CD3+CD25+) were not decreased significantly by 5-FU treatment (from 65±21 cells/mm³ to 37±10), however, the levels were dramatically increased 12 and 20 days after high dose TA1 treatment (297±136 and 321±75 cells/mm³ vs 166±70 and 212±77 cells/mm³, respectively). TA1 provided by continuous infusion lead to an even greater increase, to 422±105 and 446±73 cells/mm³.

### EXAMPLE 2

10 week old rats, weighing 250-300 g, received 100 μg/kg 5-FU for immune suppression.

8 days after 5-FU treatment, rats were randomly assigned to one of the following groups (n=15):
Control (saline in minipump)
High dose TA1, injected s.c. at 3.5 mg/Kg (with empty minipumps)
Continuous infusion TA1, provided by minipump at 3.5 mg/Kg/5 days

Immune parameters were determined at baseline and 8 days after 5-FU treatment (day 1 of TA1 treatment), and also at 5 and 14 days after TA1 treatment.

The evaluations included total leukocyte number (judged by physical cytofluorimetric parameters, after verifying the specificity by monoclonal antibody), granulocytes (flow cytometry using FITC anti rat granulocyte HIS-48), total lymphocyte number (CD3+ by flow cytometry), T helper lymphocytes (CD4+ by flow cytometry), activated lymphocytes (CD25+CD3+ by flow cytometry), and cytokine expression in plasma (IL-2 and IFN-γ by ELISA).

After determining in Example 1 that TA1 provided by continuous infusion compared to s.c. injection had a dramatic effect on the total number of leukocytes, it was of interest to determine which type of white blood cell was responsible for the increase. Granulocytes appear to be the subset of white blood cells that are most affected by TA1 provided by continuous infusion. The number of granulocytes was decreased after 5-FU from 4,485±1,116 to 1,249±432. Treatment with TA1 resulted in an increase to 14,652±2,463 within 5 days (compared to 9,924±3,218 with TA1 by injection or 6,954±1,519 with no TA1), and this level was still the highest after 14 days.

Interestingly, there was one animal in this study which was provided TA1 by BOTH injection (of 3.5 mg/Kg) and by continuous infusion (of another 3.5 mg/Kg). Not only was this animal healthy and vigorous, with no obvious adverse events, but the TA1 effects on the immune parameters measured were even greater than those in the other animals. For granulocytes, this study animal had a greatly increased level of 19,376 cells/mm³ after 5 days, compared to the mean of 14,652±2,463 in the other infused animals.

The number of total lymphocytes (CD3+) was dramatically decreased by 5-FU treatment (from 10,904±1,973 cells/mm³ to 1,740±560). Treatment with TAI allowed for a recovery to baseline levels, which occurred after only 5 days when TA1 was provided by continuous infusion but was not seen until 14 days for injected TA1.

The animal that had TA1 provided by both injection and infusion had levels of lymphocytes which were not much different from the other animals (9,765 cells/mm³ compared to the mean of 9,644±961), but the percentage of these lymphocytes which were activated was greatly increased (from 428±89, or 4% of lymphocytes, for the animals with TA1 by infusion, to 976, or 10% of lymphocytes, for the animal which had TA1 in a high dose injection followed by infusion). T helper lymphocytes (CD3+CD4+) were also depressed by treatment with 5-FU, from 5,411±1,084 cells/mm³ to 1,710±449. These depressed levels of T cells did not increase without treatment with TA1 for the 14 days of the experiment. By contrast with the results seen for granulocytes, in which TA1 provided by continuous infusion was superior to TA1 provided by injection for recovery of cell numbers, TA1 provided by either delivery method was sufficient to return the levels of T helper cells to baseline.

Since TA1 provided either by injection or by continuous infusion lead to an increase in CD4+ T helper lymphocytes, it was of interest to determine whether this increase was due to an effect on the Th1 or the Th2 subset of T helper cells. Previous in vitro and in vivo data have demonstrated that TA1 increases the Th1 subset of T cells, and in this study the same effect was seen. Providing TA1 by continuous infusion lead to an even greater increase in the plasma level of the Th1 cytokine IL-2 than was seen after s.c. injection (42±7 pg/ml 14 days after TA1 by continuous infusion, compared to 21±16 for injected TA1 and 10±16 for control animals).

Treatment by TA1 lead to an increase in the Th1 cytokine IL-2, and TA1 allows for an increase in another Th1 cytokine, IFN-γ. Although the levels are low, by 5 days after treatment, s.c. injected TA1 lead to higher plasma levels of IFN-γ. By 14 days after treatment the animals with TA1 provided by continuous infusion had the highest levels (14±5 pg/ml compared to 10±1 by injection or 8±8 for control).

The animal which received TA1 by both injection and continuous infusion had even greater levels of both of the Th1 cytokines measured, especially IFN-γ, which was 45 pg/ml after 14 days, compared to 14±5 pg/ml for the other animals.

### CONCLUSIONS

Maintenance of a constant level of TA 1 over a plurality of days in the circulation increases the measured immunological effects.

This dosage regimen leads to unexpected positive effects on granulocytes, as well as the positive effects on monocytes seen after injection of TA1.

No adverse events were observed, even at doses of TA1 15 times higher than usual (and in one animal, at doses 30 times higher than usual).

We claim:

1. A method of administering a Thymosin alpha 1 (TA1) peptide to a patient in need of immune stimulation, comprising administering to said patient said TA1 peptide so as to substantially continuously maintain an immune stimulating-effective amount of the TA1 peptide in the patient during a treatment period of at least about 6 hours.

2. The method of claim 1 wherein said treatment period is at least about 8 hours.

3. The method of claim 1 wherein said treatment period is at least about 10 hours.

4. The method of claim 1 wherein said treatment period is at least about 12 hours.

5. The method of claim 1 wherein said treatment period is at least about one day.

6. The method of claim 1 wherein said treatment period comprises a plurality of days.

7. The method of claim 1 wherein said TA1 peptide is TA1.

8. The method of claim 1 wherein said TA1 peptide is present in a pharmaceutically acceptable liquid carrier, and is substantially continuously infused into said patient during said treatment period.

9. The method of claim 8 wherein said treatment period is at least about 8 hours.

10. The method of claim 8 wherein said treatment period is at least about 10 hours.

11. The method of claim 8 wherein said treatment period is at least about 12 hours.

12. The method of claim 8 wherein said treatment period is at least about one day.

13. The method of claim 8 wherein said treatment period comprises a plurality of days.

14. The method of claim 8 wherein said TA1 peptide is TA1 (SEQ ID NO:1).

15. The method of claim 14 wherein said TA1 is administered to said patient at a rate within a range of about 0.0001-0.1 mg/hr/Kg patient body weight.

16. The method of claim 14 wherein said range is about 0.0003-0.03 mg/hr/Kg patient body weight.

17. A method of administering a Thymosin alpha 1 (TA1) peptide to a patient in need of immune stimulation, comprising substantially continuously infusing an immune stimulating-effective amount of said TA1 peptide into the patient for a period of at least about one hour.

18. The method of claim 17 wherein said TA1 peptide is TA1, and wherein said TA1 is continuously infused into said patient for a period of at least about 6 hours.

19. The method of claim 17 wherein said TA1 is continuously infused into said patient for at least about one day.

20. The method of claim 17 wherein said TA1 is continuously infused into said patient for a plurality of days.
